(19) United States
(12) Patent Application Publication
(10) Pub. No.: US 2022/0257522 A1
(43) Pub. Date: Aug. 18, 2022

(54) DOSE DUMPING RESISTANT PHARMACEUTICAL COMPOSITIONS COMPRISING VERINURAD

(71) Applicant: AstraZeneca AB, Södertälje (SE)

(72) Inventors: Christian von Corswant, Södertälje (SE); Laleh Maleki, Södertälje (SE)

(21) Appl. No.: 17/597,601

(22) PCT Filed: Jul. 15, 2020
(86) PCT No.: PCT/EP2020/069941

§ 371 Date: Jan. 13, 2022

(51) Int. Cl. A61K 9/50; A61K 31/4418; A61K 31/519

(52) U.S. Cl. CPC A61K 9/5078; A61K 31/4418; A61K 31/519; A61K 9/5026; A61K 9/5042; A61K 9/5036

(57) ABSTRACT

Disclosed herein are pharmaceutical formulations comprising verinurad or a pharmaceutically acceptable salt thereof that are resistant to alcohol-induced dose dumping and may be used in therapeutic and/or prophylactic methods.

### BACKGROUND

Verinurad has the chemical name 2-((3-(4-cyanonaphthalen-1-yl)pyridin-4-yl)thio)-2-methylpropanoic acid and the following structure:

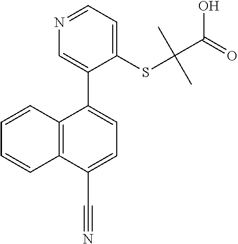 [SMILES: CC(C)(Sc1ccncc1-c1ccc(C#N)c2ccccc12)C(=O)O]

Methods of synthesizing verinurad and verinurad's activity as an inhibitor of urate transporter 1 (URAT1) inhibitor are described in International Publication No. WO 2011/159839. As an inhibitor of URAT1, verinurad is useful for the treatment or prevention of diseases or medical conditions mediated alone or in part by elevated serum uric acid (sUA) levels.

Alcohol-induced dose dumping (ADD) is the premature and/or exaggerated release of an active pharmaceutical agent from an orally administered pharmaceutical composition, which is caused by ingestion of ethanol by a patient while the patient is receiving treatment with the pharmaceutical composition. ADD can expose patients to dangerously high levels of active pharmaceutical agents, potentially resulting in adverse effects and/or drug-induced toxicity. Thus, a need exists for pharmaceutical compositions comprising verinurad or a pharmaceutically acceptable salt thereof that are resistant to ADD and can be used in therapeutic or prophylactic methods.

### BRIEF SUMMARY

The foregoing needs are met by the pharmaceutical compositions described herein. In particular, disclosed herein is a pharmaceutical composition, wherein the pharmaceutical composition is a multiparticulate composition comprising a plurality of pellets, wherein each pellet comprises: a core; an API layer on the core, wherein the API layer comprises verinurad or a pharmaceutically acceptable salt thereof; a rate-controlling polymer layer on the API layer; and a sodium alginate layer on the release-rate controlling polymer layer.

In some embodiments, the rate-controlling polymer layer comprises ethyl cellulose. In some embodiments, the rate-controlling polymer layer comprises polyvinylpyrrolidone. In some embodiments, the rate-controlling polymer layer comprises hydroxypropyl cellulose. In some embodiments, the core comprises microcrystalline cellulose. In some embodiments, the API layer comprises verinurad. In some embodiments, the API layer further comprises hydroxypropyl methylcellulose. In some embodiments, the API layer further comprises a xanthine oxidase inhibitor. In some embodiments, the xanthine oxidase inhibitor is allopurinol. In some embodiments, the sodium alginate has a glucuronic acid content between 65% and 75%, and a mannuronic acid content between 25% and 35%. In some embodiments, the amount of sodium alginate layer is between 15 wt % and 25 wt %.

In some embodiments disclosed herein are methods of reducing serum uric acid levels in a human comprising administering a pharmaceutical composition disclosed herein to the human. In some embodiments disclosed herein are methods of treating or preventing hyperuricemia, gout, gouty arthritis, recurrent gout attacks, polycythemia, myeloid metaplasia, inflammatory arthritis, nephrolithiasis (kidney stones), joint inflammation, urolithiasis (formation of calculus in the urinary tract), deposition of urate crystals in joints, deposition of urate crystals in renal parenchyma, plumbism, hyperparathyroidism, psoriasis, sarcoidosis, Lesch-Nyhan syndrome, Kelley-Seegmiller syndrome, gout flare, tophaceous gout, chronic kidney disease, kidney failure, heart failure, hypertension, cardiovascular disease, coronary heart disease, or a combination thereof in a human comprising administering a pharmaceutical composition disclosed herein to the human.

In some embodiments, disclosed herein is a method of treating or preventing chronic kidney disease in a human comprising administering a pharmaceutical composition disclosed herein to the human. In some embodiments, disclosed herein is a method of treating or preventing heart failure in a human comprising administering a pharmaceutical composition disclosed herein to the human.

### BRIEF DESCRIPTION OF THE DRAWINGS

FIG. 1 shows the ratio of released amount of verinurad in 20% ethanol medium relative to a medium with 0% ethanol from various example formulations comprising different sodium alginate grades at 25 wt % layer coating.

FIG. 2 shows the ratio of released amount of verinurad in 20% ethanol medium relative to medium with 0% ethanol from various example formulations comprising different amounts of an LFR 5/60 sodium alginate layer.

### DETAILED DESCRIPTION

While embodiments of the invention are shown and described herein, it will be apparent to those skilled in the art that such embodiments are provided by way of example only. Numerous variations, changes, and substitutions will occur to those skilled in the art without departing from the invention. It should be understood that various alternatives to the embodiments described herein may be employed. The section headings used herein are for organizational purposes only and are not to be construed as limiting the subject matter described.

### Definitions

The term “pharmaceutical composition,” as used herein, refers to a composition of matter comprising verinurad or a pharmaceutically acceptable salt thereof and at least one pharmaceutically acceptable excipient, including but not limited to carriers, stabilizers, diluents, dispersing agents, suspending agents, thickening agents, and the like.

The terms “treat,” “treating,” or “treatment,” and other grammatical equivalents as used herein, include alleviating, abating or ameliorating a disease or condition or one or more symptoms thereof, ameliorating the underlying metabolic causes of symptoms, inhibiting the disease or condition, relieving the disease or condition, causing regression of the disease or condition, relieving a condition caused by the disease or condition, or stopping the symptoms of the disease or condition.

The terms “administer,” “administering,” “administration,” and their grammatical equivalents, as used herein, refer to the methods used to deliver pharmaceutical compositions disclosed herein to the desired site of biological action.

The terms “co-administration”, “administered in combination with” and their grammatical equivalents, as used herein, are meant to encompass administration of the pharmaceutical compositions disclosed herein to a single individual, and, unless specified otherwise, include treatment regimens in which the agents are administered by the same or different route of administration or at the same or different times. They include simultaneous administration in separate compositions, administration at different times in separate compositions, or administration in a composition in which one or more therapeutic agents are present.

The term “pharmaceutically acceptable,” as used herein, refers to a material, such as a carrier or diluent, which does not abrogate the biological activity or properties of verinurad, and is relatively nontoxic, i.e., the material may be administered to an individual without causing undesirable biological effects or interacting in a deleterious manner with any of the components of the composition in which it is contained.

The term “pharmaceutically acceptable salt,” as used herein, refers to salts that retain the biological efficacy of the free acid and base of verinurad and that are not biologically or otherwise undesirable. Verinurad may react with inorganic or organic bases, and inorganic and organic acids, to form a pharmaceutically acceptable salt. These salts can be prepared in situ during the final isolation and purification, or by separately reacting the purified compound in its free base form with a suitable organic or inorganic acid, and isolating the salt thus formed.

### Formulations

In some embodiments, disclosed herein is a pharmaceutical composition, wherein the pharmaceutical composition is a multiparticulate composition comprising a plurality of pellets, wherein each pellet comprises: a core; an API layer on the core, wherein the API layer comprises verinurad or a pharmaceutically acceptable salt thereof; a rate-controlling polymer layer on the API layer; and a sodium alginate layer on the release-rate controlling polymer layer.

In some embodiments, the core comprises microcrystalline cellulose. In some embodiments, the API layer comprises verinurad. In some embodiments, the API layer further comprises hydroxypropyl methylcellulose.

In some embodiments, the rate-controlling polymer layer comprises ethyl cellulose. In some embodiments, the rate-controlling polymer layer comprises polyvinylpyrrolidone. In some embodiments, the rate-controlling polymer layer comprises hydroxypropyl cellulose. In some embodiments, the rate-controlling polymer layer comprises ethyl cellulose and polyvinylpyrrolidone. In some embodiments, the rate-controlling polymer layer comprises ethyl cellulose and hydroxypropyl cellulose.

In some embodiments, the sodium alginate layer of the pharmaceutical compositions disclosed herein has a glucuronic acid content between 50% and 75%, and a mannuronic acid content between 25% and 50%. In some embodiments, the sodium alginate layer of the pharmaceutical compositions disclosed herein has a glucuronic acid content between 55% and 75%, and a mannuronic acid content between 25% and 45%. In some embodiments, the sodium alginate layer of the pharmaceutical compositions disclosed herein has a glucuronic acid content between 60% and 75%, and a mannuronic acid content between 25% and 40%. In some embodiments, the sodium alginate layer of the pharmaceutical compositions disclosed herein has a glucuronic acid content between 50% and 70%, and a mannuronic acid content between 30% and 50%. In some embodiments, the sodium alginate layer of the pharmaceutical compositions disclosed herein has a glucuronic acid content between 55% and 70%, and a mannuronic acid content between 30% and 45%. In some embodiments, the sodium alginate layer of the pharmaceutical compositions disclosed herein has a glucuronic acid content between 50% and 65%, and a mannuronic acid content between 35% and 50%. In some embodiments, the sodium alginate layer of the pharmaceutical compositions disclosed herein has a glucuronic acid content between 50% and 60%, and a mannuronic acid content between 40% and 50%. In some embodiments, the sodium alginate layer of the pharmaceutical compositions disclosed herein has a glucuronic acid content between 60% and 70%, and a mannuronic acid content between 30% and 40%.

In some embodiments, the sodium alginate layer of the pharmaceutical compositions disclosed herein comprises PROTANAL® LFR 5/60. PROTANAL® LFR 5/60 has a glucuronic acid content between 65% and 75%, and a mannuronic acid content between 25% and 35%. A 10% aqueous solution of PROTANAL® LFR 5/60 at a temperature of 20° C. has a viscosity of 300-700 MPas as measured at a shear rate of 20 rpm by use of a Brookfield viscometer with spindle no. 2.

In various embodiments pharmaceutical compositions disclosed herein comprises different amounts of sodium alginate. The amount of sodium alginate in the pharmaceutical compositions is expressed herein as weight percent (wt %), which is the percent by weight of the entire pharmaceutical composition. In some embodiments, the amount of sodium alginate layer is between 10 wt % and 30 wt %. In some embodiments, the amount of sodium alginate layer is between 15 wt % and 30 wt %. In some embodiments, the amount of sodium alginate layer is between 15 wt % and 25 wt %. In some embodiments, the amount of sodium alginate layer is between 15 wt % and 20 wt %. In some embodiments, the amount of sodium alginate layer is between 20 wt % and 25 wt %. In some embodiments, the amount of sodium alginate layer is 15 wt %. In some embodiments, the amount of sodium alginate layer is 20 wt %. In some embodiments, the amount of sodium alginate layer is 25 wt %.

In some embodiments, the amount of sodium alginate layer is between 10 wt % and 30 wt % of the total weight of the plurality of pellets. In some embodiments, the amount of sodium alginate layer is between 15 wt % and 30 wt % of the total weight of the plurality of pellets. In some embodiments, the amount of sodium alginate layer is between 15 wt % and 25 wt % of the total weight of the plurality of pellets. In some embodiments, the amount of sodium alginate layer is between 15 wt % and 20 wt % of the total weight of the plurality of pellets. In some embodiments, the amount of sodium alginate layer is between 20 wt % and 25 wt % of the total weight of the plurality of pellets. In some embodiments, the amount of sodium alginate layer is 15 wt % of the total weight of the plurality of pellets. In some embodiments, the amount of sodium alginate layer is 20 wt % of the total weight of the plurality of pellets. In some embodiments, the amount of sodium alginate layer is 25 wt % of the total weight of the plurality of pellets.

In some embodiments, the core is between 40 wt % and 75 wt % of the total weight of the plurality of pellets. In some embodiments, the core is between 50 wt % and 70 wt % of the total weight of the plurality of pellets. In some embodiments, the core is between 50 wt % and 60 wt % of the total weight of the plurality of pellets.

In various embodiments the core comprises microcrystalline cellulose. In some embodiments, the amount of microcrystalline cellulose is between 40 wt % and 75 wt % of the total weight of the plurality of pellets. In some embodiments, the amount of microcrystalline cellulose is between 50 wt % and 70 wt % of the total weight of the plurality of pellets. In some embodiments, the amount of microcrystalline cellulose is between 50 wt % and 60 wt % of the total weight of the plurality of pellets.

In some embodiments, the API layer is between 1 wt % and 10 wt % of the total weight of the plurality of pellets. In some embodiments, the API layer is between 3 wt % and 5 wt % of the total weight of the plurality of pellets.

In various embodiments the API layer comprises verinurad and hydroxypropyl methylcellulose. In some embodiments, the amount of verinurad is between 3 wt % and 5 wt % of the total weight of the plurality of pellets. In some embodiments, the amount of hydroxypropyl methylcellulose is between 0.3 wt % and 0.5 wt % of the total weight of the plurality of pellets.

In some embodiments, the rate-controlling polymer layer is between 10 wt % and 30 wt % of the total weight of the plurality of pellets.

In various embodiments the rate-controlling polymer layer comprises ethyl cellulose and polyvinylpyrrolidone. In some embodiments, the amount of ethyl cellulose is between 8 wt % and 18 wt % of the total weight of the plurality of pellets. In some embodiments, the amount of polyvinylpyrrolidone is between 3 wt % and 8 wt % of the total weight of the plurality of pellets.

In some embodiments, disclosed herein is a pharmaceutical composition, wherein the pharmaceutical composition is a multiparticulate composition comprising a plurality of pellets, wherein each pellet comprises: a core comprising microcrystalline cellulose; an API layer on the core, wherein the API layer comprises verinurad or a pharmaceutically acceptable salt thereof and hydroxypropyl methylcellulose; a rate-controlling polymer layer on the API layer, wherein the rate-controlling polymer layer comprises ethyl cellulose; and a sodium alginate layer on the release-rate controlling polymer layer, wherein the sodium alginate has a glucuronic acid content between 65% and 75% and a mannuronic acid content between 25% and 35%, and the amount of sodium alginate is between 15 wt % and 25 wt %.

In some embodiments, disclosed herein is a pharmaceutical composition, wherein the pharmaceutical composition is a multiparticulate composition comprising a plurality of pellets, wherein each pellet comprises: a core comprising microcrystalline cellulose; an API layer on the core, wherein the API layer comprises verinurad or a pharmaceutically acceptable salt thereof and hydroxypropyl methylcellulose; a rate-controlling polymer layer on the API layer, wherein the rate-controlling polymer layer comprises ethyl cellulose and polyvinylpyrrolidone; and a sodium alginate layer on the release-rate controlling polymer layer, wherein the sodium alginate has a glucuronic acid content between 65% and 75% and a mannuronic acid content between 25% and 35%, and the amount of sodium alginate is between 15 wt % and 25 wt %.

In some embodiments, disclosed herein is a pharmaceutical composition, wherein the pharmaceutical composition is a multiparticulate composition comprising a plurality of pellets, wherein each pellet comprises: a core comprising microcrystalline cellulose; an API layer on the core, wherein the API layer comprises verinurad or a pharmaceutically acceptable salt thereof and hydroxypropyl methylcellulose; a rate-controlling polymer layer on the API layer, wherein the rate-controlling polymer layer comprises ethyl cellulose and hydroxypropyl cellulose; and a sodium alginate layer on the release-rate controlling polymer layer, wherein the sodium alginate has a glucuronic acid content between 65% and 75% and a mannuronic acid content between 25% and 35%, and the amount of sodium alginate is between 15 wt % and 25 wt %.

In some embodiments, disclosed herein is a pharmaceutical composition, wherein the pharmaceutical composition is a multiparticulate composition comprising a plurality of pellets, wherein each pellet comprises: a core comprising microcrystalline cellulose; an API layer on the core, wherein the API layer comprises verinurad and hydroxypropyl methylcellulose; a rate-controlling polymer layer on the API layer, wherein the rate-controlling polymer layer comprises ethyl cellulose; and a sodium alginate layer on the release-rate controlling polymer layer, wherein the sodium alginate has a glucuronic acid content between 65% and 75% and a mannuronic acid content between 25% and 35%, and the amount of sodium alginate is between 15 wt % and 25 wt %.

In some embodiments, disclosed herein is a pharmaceutical composition, wherein the pharmaceutical composition is a multiparticulate composition comprising a plurality of pellets, wherein each pellet comprises: a core comprising microcrystalline cellulose; an API layer on the core, wherein the API layer comprises verinurad and hydroxypropyl methylcellulose; a rate-controlling polymer layer on the API layer, wherein the rate-controlling polymer layer comprises ethyl cellulose and polyvinylpyrrolidone; and a sodium alginate layer on the release-rate controlling polymer layer, wherein the sodium alginate has a glucuronic acid content between 65% and 75% and a mannuronic acid content between 25% and 35%, and the amount of sodium alginate is between 15 wt % and 25 wt %.

In some embodiments, disclosed herein is a pharmaceutical composition, wherein the pharmaceutical composition is a multiparticulate composition comprising a plurality of pellets, wherein each pellet comprises: a core comprising microcrystalline cellulose; an API layer on the core, wherein the API layer comprises verinurad and hydroxypropyl methylcellulose; a rate-controlling polymer layer on the API layer, wherein the rate-controlling polymer layer comprises ethyl cellulose and hydroxypropyl cellulose; and a sodium alginate layer on the release-rate controlling polymer layer, wherein the sodium alginate has a glucuronic acid content between 65% and 75% and a mannuronic acid content between 25% and 35%, and the amount of sodium alginate is between 15 wt % and 25 wt %.

In some embodiments, disclosed herein is a pharmaceutical composition, wherein the pharmaceutical composition is a multiparticulate composition comprising a plurality of pellets, wherein each pellet comprises: a core comprising microcrystalline cellulose; an API layer on the core, wherein the API layer comprises verinurad and hydroxypropyl methylcellulose; a rate-controlling polymer layer on the API layer, wherein the rate-controlling polymer layer comprises ethyl cellulose; and a sodium alginate layer on the release-rate controlling polymer layer, wherein the sodium alginate layer comprises PROTANAL® LFR 5/60.

In some embodiments, disclosed herein is a pharmaceutical composition, wherein the pharmaceutical composition is a multiparticulate composition comprising a plurality of pellets, wherein each pellet comprises: a core comprising microcrystalline cellulose; an API layer on the core, wherein the API layer comprises verinurad and hydroxypropyl methylcellulose; a rate-controlling polymer layer on the API layer, wherein the rate-controlling polymer layer comprises ethyl cellulose and polyvinylpyrrolidone; and a sodium alginate layer on the release-rate controlling polymer layer, wherein the sodium alginate layer comprises PROTANAL® LFR 5/60.

In some embodiments, disclosed herein is a pharmaceutical composition, wherein the pharmaceutical composition is a multiparticulate composition comprising a plurality of pellets, wherein each pellet comprises: a core comprising microcrystalline cellulose; an API layer on the core, wherein the API layer comprises verinurad and hydroxypropyl methylcellulose; a rate-controlling polymer layer on the API layer, wherein the rate-controlling polymer layer comprises ethyl cellulose and hydroxypropyl cellulose; and a sodium alginate layer on the release-rate controlling polymer layer, wherein the sodium alginate layer comprises PROTANAL® LFR 5/60.

In some embodiments, disclosed herein is a pharmaceutical composition, wherein the pharmaceutical composition is a multiparticulate composition comprising a plurality of pellets, wherein each pellet comprises: a core comprising microcrystalline cellulose; an API layer on the core, wherein the API layer comprises verinurad and hydroxypropyl methylcellulose; a rate-controlling polymer layer on the API layer, wherein the rate-controlling polymer layer comprises ethyl cellulose; and a sodium alginate layer on the release-rate controlling polymer layer, wherein the sodium alginate layer comprises PROTANAL® LFR 5/60 and the amount of sodium alginate is between 15 wt % and 25 wt %.

In some embodiments, disclosed herein is a pharmaceutical composition, wherein the pharmaceutical composition is a multiparticulate composition comprising a plurality of pellets, wherein each pellet comprises: a core comprising microcrystalline cellulose; an API layer on the core, wherein the API layer comprises verinurad and hydroxypropyl methylcellulose; a rate-controlling polymer layer on the API layer, wherein the rate-controlling polymer layer comprises ethyl cellulose and polyvinylpyrrolidone; and a sodium alginate layer on the release-rate controlling polymer layer, wherein the sodium alginate layer comprises PROTANAL® LFR 5/60 and the amount of sodium alginate is between 15 wt % and 25 wt %.

In some embodiments, disclosed herein is a pharmaceutical composition, wherein the pharmaceutical composition is a multiparticulate composition comprising a plurality of pellets, wherein each pellet comprises: a core comprising microcrystalline cellulose; an API layer on the core, wherein the API layer comprises verinurad and hydroxypropyl methylcellulose; a rate-controlling polymer layer on the API layer, wherein the rate-controlling polymer layer comprises ethyl cellulose and hydroxypropyl cellulose; and a sodium alginate layer on the release-rate controlling polymer layer, wherein the sodium alginate layer comprises PROTANAL® LFR 5/60 and the amount of sodium alginate is between 15 wt % and 25 wt %.

In some embodiments, disclosed herein is a pharmaceutical composition, wherein the pharmaceutical composition is a multiparticulate composition comprising a plurality of pellets, wherein each pellet comprises: a core comprising microcrystalline cellulose; an API layer on the core, wherein the API layer comprises verinurad and hydroxypropyl methylcellulose; a rate-controlling polymer layer on the API layer, wherein the rate-controlling polymer layer comprises ethyl cellulose; and a sodium alginate layer on the release-rate controlling polymer layer, wherein the sodium alginate layer comprises PROTANAL® LFR 5/60 and the amount of sodium alginate is 15 wt %.

In some embodiments, disclosed herein is a pharmaceutical composition, wherein the pharmaceutical composition is a multiparticulate composition comprising a plurality of pellets, wherein each pellet comprises: a core comprising microcrystalline cellulose; an API layer on the core, wherein the API layer comprises verinurad and hydroxypropyl methylcellulose; a rate-controlling polymer layer on the API layer, wherein the rate-controlling polymer layer comprises ethyl cellulose and polyvinylpyrrolidone; and a sodium alginate layer on the release-rate controlling polymer layer, wherein the sodium alginate layer comprises PROTANAL® LFR 5/60 and the amount of sodium alginate is 15 wt %.

In some embodiments, disclosed herein is a pharmaceutical composition, wherein the pharmaceutical composition is a multiparticulate composition comprising a plurality of pellets, wherein each pellet comprises: a core comprising microcrystalline cellulose; an API layer on the core, wherein the API layer comprises verinurad and hydroxypropyl methylcellulose; a rate-controlling polymer layer on the API layer, wherein the rate-controlling polymer layer comprises ethyl cellulose and hydroxypropyl cellulose; and a sodium alginate layer on the release-rate controlling polymer layer, wherein the sodium alginate layer comprises PROTANAL® LFR 5/60 and the amount of sodium alginate is 15 wt %.

In some embodiments, disclosed herein is a pharmaceutical composition, wherein the pharmaceutical composition is a multiparticulate composition comprising a plurality of pellets, wherein each pellet comprises: a core comprising microcrystalline cellulose; an API layer on the core, wherein the API layer comprises verinurad and hydroxypropyl methylcellulose; a rate-controlling polymer layer on the API layer, wherein the rate-controlling polymer layer comprises ethyl cellulose; and a sodium alginate layer on the release-rate controlling polymer layer, wherein the sodium alginate layer comprises PROTANAL® LFR 5/60 and the amount of sodium alginate is 20 wt %.

In some embodiments, disclosed herein is a pharmaceutical composition, wherein the pharmaceutical composition is a multiparticulate composition comprising a plurality of pellets, wherein each pellet comprises: a core comprising microcrystalline cellulose; an API layer on the core, wherein the API layer comprises verinurad and hydroxypropyl methylcellulose; a rate-controlling polymer layer on the API layer, wherein the rate-controlling polymer layer comprises ethyl cellulose and polyvinylpyrrolidone; and a sodium alginate layer on the release-rate controlling polymer layer, wherein the sodium alginate layer comprises PROTANAL® LFR 5/60 and the amount of sodium alginate is 20 wt %.

In some embodiments, disclosed herein is a pharmaceutical composition, wherein the pharmaceutical composition is a multiparticulate composition comprising a plurality of pellets, wherein each pellet comprises: a core comprising microcrystalline cellulose; an API layer on the core, wherein the API layer comprises verinurad and hydroxypropyl methylcellulose; a rate-controlling polymer layer on the API layer, wherein the rate-controlling polymer layer comprises ethyl cellulose and hydroxypropyl cellulose; and a sodium alginate layer on the release-rate controlling polymer layer, wherein the sodium alginate layer comprises PROTANAL® LFR 5/60 and the amount of sodium alginate is 20 wt %.

In some embodiments, disclosed herein is a pharmaceutical composition, wherein the pharmaceutical composition is a multiparticulate composition comprising a plurality of pellets, wherein each pellet comprises: a core comprising microcrystalline cellulose; an API layer on the core, wherein the API layer comprises verinurad and hydroxypropyl methylcellulose; a rate-controlling polymer layer on the API layer, wherein the rate-controlling polymer layer comprises ethyl cellulose; and a sodium alginate layer on the release-rate controlling polymer layer, wherein the sodium alginate layer comprises PROTANAL® LFR 5/60 and the amount of sodium alginate is 25 wt %.

In some embodiments, disclosed herein is a pharmaceutical composition, wherein the pharmaceutical composition is a multiparticulate composition comprising a plurality of pellets, wherein each pellet comprises: a core comprising microcrystalline cellulose; an API layer on the core, wherein the API layer comprises verinurad and hydroxypropyl methylcellulose; a rate-controlling polymer layer on the API layer, wherein the rate-controlling polymer layer comprises ethyl cellulose and polyvinylpyrrolidone; and a sodium alginate layer on the release-rate controlling polymer layer, wherein the sodium alginate layer comprises PROTANAL® LFR 5/60 and the amount of sodium alginate is 25 wt %.

In some embodiments, disclosed herein is a pharmaceutical composition, wherein the pharmaceutical composition is a multiparticulate composition comprising a plurality of pellets, wherein each pellet comprises: a core comprising microcrystalline cellulose; an API layer on the core, wherein the API layer comprises verinurad and hydroxypropyl methylcellulose; a rate-controlling polymer layer on the API layer, wherein the rate-controlling polymer layer comprises ethyl cellulose and hydroxypropyl cellulose; and a sodium alginate layer on the release-rate controlling polymer layer, wherein the sodium alginate layer comprises PROTANAL® LFR 5/60 and the amount of sodium alginate is 25 wt %.

In some embodiments, disclosed herein is a pharmaceutical composition, wherein the pharmaceutical composition is a multiparticulate composition comprising a plurality of pellets, wherein each pellet comprises: a core comprising microcrystalline cellulose; an API layer on the core, wherein the API layer comprises verinurad or a pharmaceutically acceptable salt thereof; a rate-controlling polymer layer on the API layer, wherein the rate-controlling polymer layer comprises ethyl cellulose and polyvinylpyrrolidone; and a sodium alginate layer on the release-rate controlling polymer layer, wherein the sodium alginate has a glucuronic acid content between 65% and 75% and and a mannuronic acid content between 25% and 35%.

In some embodiments, disclosed herein is a pharmaceutical composition, wherein the pharmaceutical composition is a multiparticulate composition comprising a plurality of pellets, wherein each pellet comprises: a core comprising microcrystalline cellulose; an API layer on the core, wherein the API layer comprises verinurad or a pharmaceutically acceptable salt thereof; a rate-controlling polymer layer on the API layer, wherein the rate-controlling polymer layer comprises ethyl cellulose and polyvinylpyrrolidone; and a sodium alginate layer on the release-rate controlling polymer layer, wherein the sodium alginate has a glucuronic acid content between 65% and 75% and a mannuronic acid content between 25% and 35%, and the amount of sodium alginate is between 15 wt % and 25 wt %.

In some embodiments, disclosed herein is a pharmaceutical composition, wherein the pharmaceutical composition is a multiparticulate composition comprising a plurality of pellets, wherein each pellet comprises: a core comprising microcrystalline cellulose; an API layer on the core, wherein the API layer comprises verinurad or a pharmaceutically acceptable salt thereof; a rate-controlling polymer layer on the API layer, wherein the rate-controlling polymer layer comprises ethyl cellulose and polyvinylpyrrolidone; and a sodium alginate layer on the release-rate controlling polymer layer, wherein the sodium alginate has a glucuronic acid content between 65% and 75% and a mannuronic acid content between 25% and 35%, and the amount of sodium alginate is 15 wt %.

In some embodiments, disclosed herein is a pharmaceutical composition, wherein the pharmaceutical composition is a multiparticulate composition comprising a plurality of pellets, wherein each pellet comprises: a core comprising microcrystalline cellulose; an API layer on the core, wherein the API layer comprises verinurad or a pharmaceutically acceptable salt thereof; a rate-controlling polymer layer on the API layer, wherein the rate-controlling polymer layer comprises ethyl cellulose and polyvinylpyrrolidone; and a sodium alginate layer on the release-rate controlling polymer layer, wherein the sodium alginate has a glucuronic acid content between 65% and 75% and a mannuronic acid content between 25% and 35%, and the amount of sodium alginate is 20 wt %.

In some embodiments, disclosed herein is a pharmaceutical composition, wherein the pharmaceutical composition is a multiparticulate composition comprising a plurality of pellets, wherein each pellet comprises: a core comprising microcrystalline cellulose; an API layer on the core, wherein the API layer comprises verinurad or a pharmaceutically acceptable salt thereof; a rate-controlling polymer layer on the API layer, wherein the rate-controlling polymer layer comprises ethyl cellulose and polyvinylpyrrolidone; and a sodium alginate layer on the release-rate controlling polymer layer, wherein the sodium alginate has a glucuronic acid content between 65% and 75% and a mannuronic acid content between 25% and 35%, and the amount of sodium alginate is 25 wt %.

In some embodiments, disclosed herein is a pharmaceutical composition, wherein the pharmaceutical composition is a multiparticulate composition comprising a plurality of pellets, wherein each pellet comprises: a core comprising microcrystalline cellulose; an API layer on the core, wherein the API layer comprises verinurad; a rate-controlling polymer layer on the API layer, wherein the rate-controlling polymer layer comprises ethyl cellulose and polyvinylpyrrolidone; and a sodium alginate layer on the release-rate controlling polymer layer, wherein the sodium alginate has a glucuronic acid content between 65% and 75%, and a mannuronic acid content between 25% and 35%.

In some embodiments, disclosed herein is a pharmaceutical composition, wherein the pharmaceutical composition is a multiparticulate composition comprising a plurality of pellets, wherein each pellet comprises: a core comprising microcrystalline cellulose; an API layer on the core, wherein the API layer comprises verinurad; a rate-controlling polymer layer on the API layer, wherein the rate-controlling polymer layer comprises ethyl cellulose and polyvinylpyrrolidone; and a sodium alginate layer on the release-rate controlling polymer layer, wherein the sodium alginate has a glucuronic acid content between 65% and 75% and a mannuronic acid content between 25% and 35%, and the amount of sodium alginate is between 15 wt % and 25 wt %.

In some embodiments, disclosed herein is a pharmaceutical composition, wherein the pharmaceutical composition is a multiparticulate composition comprising a plurality of pellets, wherein each pellet comprises: a core comprising microcrystalline cellulose in an amount of between 40 wt % and 75 wt % of the total weight of the plurality of pellets; an API layer on the core, wherein the API layer comprises verinurad in an amount of between 3 wt % and 5 wt % of the total weight of the plurality of pellets and optionally hydroxypropyl methylcellulose in an amount of between 0.3 wt % and 0.5 wt % of the total weight of the plurality of pellets; a rate-controlling polymer layer on the API layer, wherein the rate-controlling polymer layer comprises ethyl cellulose in an amount of between 8 wt % and 18 wt % of the total weight of the plurality of pellets and polyvinylpyrrolidone in an amount of between 3 wt % and 8 wt % of the total weight of the plurality of pellets; and a sodium alginate layer on the release-rate controlling polymer layer, wherein the sodium alginate has a glucuronic acid content between 65% and 75% and a mannuronic acid content between 25% and 35%, and the amount of sodium alginate is between 15 wt % and 30 wt % of the total weight of the plurality of pellets.

In some embodiments, disclosed herein is a pharmaceutical composition, wherein the pharmaceutical composition is a multiparticulate composition comprising a plurality of pellets, wherein each pellet comprises: a core comprising microcrystalline cellulose in an amount of between 40 wt % and 80 wt % of the total weight of the plurality of pellets; an API layer on the core, wherein the API layer comprises verinurad in an amount of between 3 wt % and 5 wt % of the total weight of the plurality of pellets and optionally hydroxypropyl methylcellulose in an amount of between 0.3 wt % and 0.5 wt % of the total weight of the plurality of pellets; a rate-controlling polymer layer on the API layer, wherein the rate-controlling polymer layer comprises ethyl cellulose in an amount of between 8 wt % and 18 wt % of the total weight of the plurality of pellets and polyvinylpyrrolidone in an amount of between 3 wt % and 8 wt % of the total weight of the plurality of pellets; and a sodium alginate layer on the release-rate controlling polymer layer, wherein the sodium alginate has a glucuronic acid content between 65% and 75% and a mannuronic acid content between 25% and 35%, and the amount of sodium alginate is 25 wt % of the total weight of the plurality of pellets.

In some embodiments, disclosed herein is a pharmaceutical composition, wherein the pharmaceutical composition is a multiparticulate composition comprising a plurality of pellets, wherein each pellet comprises: a core comprising microcrystalline cellulose; an API layer on the core, wherein the API layer comprises verinurad; a rate-controlling polymer layer on the API layer, wherein the rate-controlling polymer layer comprises ethyl cellulose and polyvinylpyrrolidone; and a sodium alginate layer on the release-rate controlling polymer layer, wherein the sodium alginate has a glucuronic acid content between 65% and 75% and a mannuronic acid content between 25% and 35%, and the amount of sodium alginate is 15 wt %.

In some embodiments, disclosed herein is a pharmaceutical composition, wherein the pharmaceutical composition is a multiparticulate composition comprising a plurality of pellets, wherein each pellet comprises: a core comprising microcrystalline cellulose; an API layer on the core, wherein the API layer comprises verinurad; a rate-controlling polymer layer on the API layer, wherein the rate-controlling polymer layer comprises ethyl cellulose and polyvinylpyrrolidone; and a sodium alginate layer on the release-rate controlling polymer layer, wherein the sodium alginate has a glucuronic acid content between 65% and 75% and a mannuronic acid content between 25% and 35%, and the amount of sodium alginate is 20 wt %.

In some embodiments, disclosed herein is a pharmaceutical composition, wherein the pharmaceutical composition is a multiparticulate composition comprising a plurality of pellets, wherein each pellet comprises: a core comprising microcrystalline cellulose; an API layer on the core, wherein the API layer comprises verinurad; a rate-controlling polymer layer on the API layer, wherein the rate-controlling polymer layer comprises ethyl cellulose and polyvinylpyrrolidone; and a sodium alginate layer on the release-rate controlling polymer layer, wherein the sodium alginate has a glucuronic acid content between 65% and 75% and a mannuronic acid content between 25% and 35%, and the amount of sodium alginate is 25 wt %.

In some embodiments, disclosed herein is a pharmaceutical composition, wherein the pharmaceutical composition is a multiparticulate composition comprising a plurality of pellets, wherein each pellet comprises: a core comprising microcrystalline cellulose; an API layer on the core, wherein the API layer comprises verinurad or a pharmaceutically acceptable salt thereof; a rate-controlling polymer layer on the API layer, wherein the rate-controlling polymer layer comprises ethyl cellulose and polyvinylpyrrolidone; and a sodium alginate layer on the release-rate controlling polymer layer, wherein the sodium alginate layer comprises PROTANAL® LFR 5/60.

In some embodiments, disclosed herein is a pharmaceutical composition, wherein the pharmaceutical composition is a multiparticulate composition comprising a plurality of pellets, wherein each pellet comprises: a core comprising microcrystalline cellulose; an API layer on the core, wherein the API layer comprises verinurad or a pharmaceutically acceptable salt thereof; a rate-controlling polymer layer on the API layer, wherein the rate-controlling polymer layer comprises ethyl cellulose and polyvinylpyrrolidone; and a sodium alginate layer on the release-rate controlling polymer layer, wherein the sodium alginate layer comprises PROTANAL® LFR 5/60, and the amount of sodium alginate is between 15 wt % and 25 wt %.

In some embodiments, disclosed herein is a pharmaceutical composition, wherein the pharmaceutical composition is a multiparticulate composition comprising a plurality of pellets, wherein each pellet comprises: a core comprising microcrystalline cellulose; an API layer on the core, wherein the API layer comprises verinurad or a pharmaceutically acceptable salt thereof; a rate-controlling polymer layer on the API layer, wherein the rate-controlling polymer layer comprises ethyl cellulose and polyvinylpyrrolidone; and a sodium alginate layer on the release-rate controlling polymer layer, wherein the sodium alginate comprises PROTANAL® LFR 5/60, and the amount of sodium alginate is 15 wt %.

In some embodiments, disclosed herein is a pharmaceutical composition, wherein the pharmaceutical composition is a multiparticulate composition comprising a plurality of pellets, wherein each pellet comprises: a core comprising microcrystalline cellulose; an API layer on the core, wherein the API layer comprises verinurad or a pharmaceutically acceptable salt thereof; a rate-controlling polymer layer on the API layer, wherein the rate-controlling polymer layer comprises ethyl cellulose and polyvinylpyrrolidone; and a sodium alginate layer on the release-rate controlling polymer layer, wherein the sodium alginate comprises PROTANAL® LFR 5/60, and the amount of sodium alginate is 20 wt %.

In some embodiments, disclosed herein is a pharmaceutical composition, wherein the pharmaceutical composition is a multiparticulate composition comprising a plurality of pellets, wherein each pellet comprises: a core comprising microcrystalline cellulose; an API layer on the core, wherein the API layer comprises verinurad or a pharmaceutically acceptable salt thereof; a rate-controlling polymer layer on the API layer, wherein the rate-controlling polymer layer comprises ethyl cellulose and polyvinylpyrrolidone; and a sodium alginate layer on the release-rate controlling polymer layer, wherein the sodium alginate comprises PROTANAL® LFR 5/60, and the amount of sodium alginate is 25 wt %.

In some embodiments, disclosed herein is a pharmaceutical composition, wherein the pharmaceutical composition is a multiparticulate composition comprising a plurality of pellets, wherein each pellet comprises: a core comprising microcrystalline cellulose; an API layer on the core, wherein the API layer comprises verinurad; a rate-controlling polymer layer on the API layer, wherein the rate-controlling polymer layer comprises ethyl cellulose and polyvinylpyrrolidone; and a sodium alginate layer on the release-rate controlling polymer layer, wherein the sodium alginate comprises PROTANAL® LFR 5/60.

In some embodiments, disclosed herein is a pharmaceutical composition, wherein the pharmaceutical composition is a multiparticulate composition comprising a plurality of pellets, wherein each pellet comprises: a core comprising microcrystalline cellulose; an API layer on the core, wherein the API layer comprises verinurad; a rate-controlling polymer layer on the API layer, wherein the rate-controlling polymer layer comprises ethyl cellulose and polyvinylpyrrolidone; and a sodium alginate layer on the release-rate controlling polymer layer, wherein the sodium alginate comprises PROTANAL® LFR 5/60, and the amount of sodium alginate is between 15 wt % and 25 wt %.

In some embodiments, disclosed herein is a pharmaceutical composition, wherein the pharmaceutical composition is a multiparticulate composition comprising a plurality of pellets, wherein each pellet comprises: a core comprising microcrystalline cellulose; an API layer on the core, wherein the API layer comprises verinurad; a rate-controlling polymer layer on the API layer, wherein the rate-controlling polymer layer comprises ethyl cellulose and polyvinylpyrrolidone; and a sodium alginate layer on the release-rate controlling polymer layer, wherein the sodium alginate comprises PROTANAL® LFR 5/60, and the amount of sodium alginate is 15 wt %.

In some embodiments, disclosed herein is a pharmaceutical composition, wherein the pharmaceutical composition is a multiparticulate composition comprising a plurality of pellets, wherein each pellet comprises: a core comprising microcrystalline cellulose; an API layer on the core, wherein the API layer comprises verinurad; a rate-controlling polymer layer on the API layer, wherein the rate-controlling polymer layer comprises ethyl cellulose and polyvinylpyrrolidone; and a sodium alginate layer on the release-rate controlling polymer layer, wherein the sodium alginate comprises PROTANAL® LFR 5/60, and the amount of sodium alginate is 20 wt %.

In some embodiments, disclosed herein is a pharmaceutical composition, wherein the pharmaceutical composition is a multiparticulate composition comprising a plurality of pellets, wherein each pellet comprises: a core comprising microcrystalline cellulose; an API layer on the core, wherein the API layer comprises verinurad; a rate-controlling polymer layer on the API layer, wherein the rate-controlling polymer layer comprises ethyl cellulose and polyvinylpyrrolidone; and a sodium alginate layer on the release-rate controlling polymer layer, wherein the sodium alginate comprises PROTANAL® LFR 5/60, and the amount of sodium alginate is 25 wt %.

In some embodiments, pharmaceutical compositions disclosed herein comprise one or more of sweetening agents, flavoring agents, coloring agents and preserving agents. In some embodiments, pharmaceutical compositions disclosed herein comprise one or more additional inert diluents, fillers, granulating agents, disintegrating agents, binding agents, and lubricating agents. Pharmaceutical compositions disclosed herein are intended for oral administration. In some embodiments, pharmaceutical compositions disclosed herein are in the form of a tablet. In some embodiments, pharmaceutical compositions disclosed herein are in the form of a capsule. In some embodiments, pharmaceutical compositions disclosed herein are in the form of a pill.

The formulations may conveniently be presented in unit dosage form and may be prepared by any of the methods known in the art. In some embodiments, pharmaceutical compositions disclosed herein are in a unit dosage form suitable for single administration of precise dosages.

In some embodiments, pharmaceutical compositions disclosed herein comprise 2 mg of verinurad or a pharmaceutically acceptable salt thereof. In some embodiments, pharmaceutical compositions disclosed herein comprise 3 mg of verinurad or a pharmaceutically acceptable salt thereof. In some embodiments, pharmaceutical compositions disclosed herein comprise 4 mg of verinurad or a pharmaceutically acceptable salt thereof. In some embodiments, pharmaceutical compositions disclosed herein comprise 5 mg of verinurad or a pharmaceutically acceptable salt thereof. In some embodiments, pharmaceutical compositions disclosed herein comprise 6 mg of verinurad or a pharmaceutically acceptable salt thereof. In some embodiments, pharmaceutical compositions disclosed herein comprise 7 mg of verinurad or a pharmaceutically acceptable salt thereof. In some embodiments, pharmaceutical compositions disclosed herein comprise 8 mg of verinurad or a pharmaceutically acceptable salt thereof. In some embodiments, pharmaceutical compositions disclosed herein comprise 9 mg of verinurad or a pharmaceutically acceptable salt thereof. In some embodiments, pharmaceutical compositions disclosed herein comprise 10 mg of verinurad or a pharmaceutically acceptable salt thereof. In some embodiments, pharmaceutical compositions disclosed herein comprise 11 mg of verinurad or a pharmaceutically acceptable salt thereof. In some embodiments, pharmaceutical compositions disclosed herein comprise 12 mg of verinurad or a pharmaceutically acceptable salt thereof.

In some embodiments, pharmaceutical compositions disclosed herein comprise 2 mg of verinurad. In some embodiments, pharmaceutical compositions disclosed herein comprise 3 mg of verinurad. In some embodiments, pharmaceutical compositions disclosed herein comprise 4 mg of verinurad. In some embodiments, pharmaceutical compositions disclosed herein comprise 5 mg of verinurad. In some embodiments, pharmaceutical compositions disclosed herein comprise 6 mg of verinurad. In some embodiments, pharmaceutical compositions disclosed herein comprise 7 mg of verinurad. In some embodiments, pharmaceutical compositions disclosed herein comprise 8 mg of verinurad. In some embodiments, pharmaceutical compositions disclosed herein comprise 9 mg of verinurad. In some embodiments, pharmaceutical compositions disclosed herein comprise 10 mg of verinurad. In some embodiments, pharmaceutical compositions disclosed herein comprise 11 mg of verinurad. In some embodiments, pharmaceutical compositions disclosed herein comprise 12 mg of verinurad.

In some embodiments, pharmaceutical compositions disclosed herein further comprise one or more additional therapeutic agents. In some embodiments, pharmaceutical compositions disclosed herein further comprise a xanthine oxidase inhibitor. In some embodiments, pharmaceutical compositions disclosed herein further comprise allopurinol. In some embodiments, pharmaceutical compositions disclosed herein further comprise febuxostat.

In some embodiments, pharmaceutical compositions disclosed herein are administered once a day. In some embodiments, pharmaceutical compositions disclosed herein administered twice a day. In some embodiments, pharmaceutical compositions disclosed herein administered three times a day.

### Diseases

Described herein are methods of treating or preventing a disease in an individual suffering from said disease comprising administering to said individual a pharmaceutical composition described herein. Also described herein are methods of preventing or delaying onset of a disease in an individual at risk for developing said disease comprising administering to said individual a pharmaceutical composition described herein to prevent or delay onset of said disease.

Further described herein are methods for the prophylaxis or treatment of any disease or disorder in which elevated levels of uric acid plays a role including, without limitation: hyperuricemia, gout, gouty arthritis, recurrent gout attacks, polycythemia, myeloid metaplasia, inflammatory arthritis, nephrolithiasis (kidney stones), joint inflammation, urolithiasis (formation of calculus in the urinary tract), deposition of urate crystals in joints, deposition of urate crystals in renal parenchyma, plumbism, hyperparathyroidism, psoriasis, sarcoidosis, Lesch-Nyhan syndrome, Kelley-Seegmiller syndrome, gout flare, tophaceous gout, chronic kidney disease, kidney failure, heart failure, hypertension, cardiovascular disease, coronary heart disease, or combinations thereof. The methods disclosed herein extend to such a use of pharmaceutical compositions disclosed herein for treating or preventing such diseases or disorders. In some embodiments, an individual treated according to a method described herein is a human. In some embodiments, a human treated with a pharmaceutical composition disclosed herein suffers from diabetes. In some embodiments, a human treated with a pharmaceutical composition disclosed herein suffers from type 2 diabetes.

### Combination Therapies

In some embodiments, a pharmaceutical composition disclosed herein is co-administered in combination with one or more additional therapies. Regardless of the disease, disorder or condition being treated, the overall benefit experienced by the individual may be additive of the therapies or the individual may experience a synergistic benefit.

In some embodiments, a pharmaceutical composition disclosed herein is administered by a different route as one or more additional therapeutic agents. In some embodiments, a pharmaceutical composition disclosed herein is administered by the same route as one or more additional therapeutic agents. A pharmaceutical composition disclosed herein may be administered concurrently (e.g., simultaneously, essentially simultaneously or within the same treatment protocol), sequentially or dosed separately relative to the one or more additional therapeutic agents.

The particular choice of the one or more additional therapeutic agent will depend upon the diagnosis of the attending physicians and their judgment of the condition of the individual and the appropriate treatment protocol. In some embodiments, the additional agent is a URAT 1 inhibitor, a xanthine oxidase inhibitor, a xanthine dehydrogenase, a xanthine oxidoreductase inhibitor, a purine nucleoside phosphorylase (PNP) inhibitor, a uric acid transporter inhibitor, a glucose transporter (GLUT) inhibitor, a GLUT-9 inhibitor, a solute carrier family 2 (facilitated glucose transporter), member 9 (SLC2A9) inhibitor, an organic anion transporter (OAT) inhibitor, an OAT-4 inhibitor, or a combination thereof. In some embodiments, the one or more additional therapeutic agents are selected from 2-((5-bromo-4-(4-cyclopropyl-1-naphthalenyl)-4H-1,2,4-triazol-3-yl)thio)acetic acid, allopurinol, febuxostat (2-(3-cyano-4-isobutoxyphenyl)-4-methyl-1,3-thiazole-5-carboxylic acid), FYX-051 (4-(5-pyridin-4-yl-1H-[1,2,4]triazol-3-yl)pyridine-2-carbonitrile), probenecid, sulfinpyrazone, benzbromarone, acetaminophen, steroids, nonsteroidal anti-inflammatory drugs (NSAIDs), adrenocorticotropic hormone (ACTH), colchicine, a glucorticoid, an adrogen, a cox-2 inhibitor, a PPAR agonist, naproxen, sevelamer, sibutmaine, troglitazone, proglitazone, another uric acid lowering agent, losartan, fibric acid, benziodarone, salisylate, anlodipine, vitamin C, dapagliflozin, and combinations thereof.

### EXAMPLES

The examples and preparations provided below further illustrate and exemplify the present invention and do not limit in any way by the scope of the invention.

### Example 1

### Preparation of Sodium Alginate-Coated Pharmaceutical Formulations

The following procedure was used to prepare the sodium alginate-coated pharmaceutical formulations described in the following tables.

### API Coating Step

Microcrystalline cellulose spheres (JRS Pharma, 0.5-0.7 mm) were used as the starting material. The API suspension used to coat the microcrystalline cellulose spheres consisted of MilliQ water, micronized verinurad (API), and HPMC 6 cps. The dry content of the suspension was 10%, 9% API, and 1% HPMC 6 cps. The API suspension was prepared by first dissolving HPMC in purified water using a magnetic stirrer overnight. Thereafter the API was added, and the suspension was stirred prior to use. The suspension was kept stirring during the coating process. The microcrystalline cellulose spheres were coated with the API suspension in bottom sprayed fluid bed equipment (Mini-Glatt (Glatt GmbH) or labCC (Graniten Engineering AB)) to produce API coated pellets. Process parameters are shown in Table 1. The API coated pellets were manufactured to an API concentration of between 57 and 58 mg/g.

### EC/PVP Coating Step

The API coated pellets prepared according to the procedure described above were coated with ethyl cellulose (EC) and polyvinylpyrrolidone (PVP) according to the following procedure. The ethanol-based solutions for the EC/PVP coating were prepared by adding EC and PVP to 95% ethanol while stirring. The materials were left overnight to dissolve. The dry content of the solutions was 6%. The API coated pellets were coated with EC and PVP in bottom sprayed fluid bed equipment (Mini-Glatt (Glatt GmbH) or labCC (Graniten Engineering AB)). Process parameters are shown in Tables la and lb. The EC/PVP coated API pellets were manufactured to a polymer concentration of between 22.5-25.0%.

### Sodium Alginate Coating Step

The water-based solutions for the sodium alginate coating step were prepared by adding alginate to MilliQ water while stirring. The materials were left overnight to dissolve. The dry content of the solutions was 3-8%. The EC/PVP coated API pellets were coated in a bottom sprayed fluid bed equipment (MiniGlatt (Glatt GmBH). The sodium alginate coated pellets were manufactured to a sodium alginate concentration of 5-25%. Process parameters are shown in Tables 1a and 1b.

### Example Formulation A

| | Quantity | |
|---|---|---|
| Components | (wt %) | Supplier |
| Verinurad | 4.50 | AstraZeneca |
| HPMC 6 cps | 0.50 | Dow |
| Micro crystalline cellulose spheres 0.5-0.7 mm | 70.0 | JRS Pharma |
| EC 10 cps | 17.6 | Dow |
| PVP K30 | 7.38 | BASF |

### Example Formulation B

| | Quantity | |
|---|---|---|
| Components | (wt %) | Supplier |
| Verinurad | 4.65 | AstraZeneca |
| HPMC 6 cps | 0.52 | Dow |
| Micro crystalline cellulose spheres 0.5-0.7 mm | 72.3 | JRS Pharma |
| EC 10 cps | 16.0 | Dow |
| PVP K30 | 6.53 | BASF |

### Example Formulation C

| | Quantity | |
|---|---|---|
| Components | (wt %) | Supplier |
| Verinurad | 3.49 | AstraZeneca |
| HPMC 6 cps | 0.39 | Dow |
| Micro crystalline cellulose spheres 0.5-0.7 mm | 54.3 | JRS Pharma |
| EC 10 cps | 11.9 | Dow |
| PVP K30 | 4.98 | BASF |
| Sodium alginate Manucol LB | 25.0 | Dupont |

### Example Formulation D

| | Quantity | |
|---|---|---|
| Components | (wt %) | Supplier |
| Verinurad | 3.49 | AstraZeneca |
| HPMC 6 cps | 0.39 | Dow |
| Micro crystalline cellulose spheres 0.5-0.7 mm | 54.3 | JRS Pharma |
| EC 10 cps | 11.9 | Dow |
| PVP K30 | 4.98 | BASF |
| Sodium alginate Protanal CR8133 | 25.0 | Dupont |

### Example Formulation E

| | Quantity | |
|---|---|---|
| Components | (wt %) | Supplier |
| Verinurad | 3.49 | AstraZeneca |
| HPMC 6 cps | 0.39 | Dow |
| Micro crystalline cellulose spheres 0.5-0.7 mm | 54.3 | JRS Pharma |
| EC 10 cps | 11.9 | Dow |
| PVP K30 | 4.98 | BASF |
| Sodium alginate Manucol DH | 25.0 | Dupont |

### Example Formulation F

| | Quantity | |
|---|---|---|
| Components | (wt %) | Supplier |
| Verinurad | 3.49 | AstraZeneca |
| HPMC 6 cps | 0.39 | Dow |
| Micro crystalline cellulose spheres 0.5-0.7 mm | 54.3 | JRS Pharma |
| EC 10 cps | 11.9 | Dow |
| PVP K30 | 4.98 | BASF |
| Sodium alginate Manucol LKX | 25.0 | Dupont |

### Example Formulation G

| | Quantity | |
|---|---|---|
| Components | (wt %) | Supplier |
| Verinurad | 3.49 | AstraZeneca |
| HPMC 6 cps | 0.39 | Dow |
| Micro crystalline cellulose spheres 0.5-0.7 | 54.3 | JRS Pharma |
| mm | | |
| EC 10 cps | 11.9 | Dow |
| PVP K30 | 4.98 | BASF |
| Sodium alginate medium viscosity | 25.0 | Sigma-Aldrich |

### Example Formulation H

| | Quantity | |
|---|---|---|
| Components | (wt %) | Supplier |
| Verinurad | 3.49 | AstraZeneca |
| HPMC 6 cps | 0.39 | Dow |
| Micro crystalline cellulose spheres 0.5-0.7 mm | 54.3 | JRS Pharma |
| EC 10 cps | 11.9 | Dow |
| PVP K30 | 4.98 | BASF |
| Sodium alginate Protanal LFR5/60 | 25.0 | Dupont |

### Example Formulation I

| | Quantity | |
|---|---|---|
| Components | (wt %) | Supplier |
| Verinurad | 4.50 | AstraZeneca |
| HPMC 6 cps | 0.50 | Dow |
| Micro crystalline cellulose spheres 0.5-0.7 mm | 70.00 | JRS Pharma |
| EC 10 cps | 17.75 | Dow |
| PVP K30 | 7.25 | BASF |

### Example Formulation J

| | Quantity | |
|---|---|---|
| Components | (wt %) | Supplier |
| Verinurad | 4.28 | AstraZeneca |
| HPMC 6 cps | 0.48 | Dow |
| Micro crystalline cellulose spheres 0.5-0.7 mm | 66.5 | JRS Pharma |
| EC 10 cps | 16.9 | Dow |
| PVP K30 | 6.89 | BASF |
| Sodium alginate Protanal LFR5/60 | 5.00 | Dupont |

### Example Formulation K

| | Quantity | |
|---|---|---|
| Components | (wt %) | Supplier |
| Verinurad | 4.05 | AstraZeneca |
| HPMC 6 cps | 0.45 | Dow |
| Micro crystalline cellulose spheres 0.5-0.7 mm | 63.0 | JRS Pharma |
| EC 10 cps | 16.0 | Dow |
| PVP K30 | 6.53 | BASF |
| Sodium alginate Protanal LFR5/60 | 10.0 | Dupont |

### Example Formulation L

| | Quantity | |
|---|---|---|
| Components | (wt %) | Supplier |
| Verinurad | 3.83 | AstraZeneca |
| HPMC 6 cps | 0.43 | Dow |
| Micro crystalline cellulose spheres 0.5-0.7 mm | 59.5 | JRS Pharma |
| EC 10 cps | 15.1 | Dow |
| PVP K30 | 6.16 | BASF |
| Sodium alginate Protanal LFR5/60 | 15.0 | Dupont |

### Example Formulation M

| | Quantity | |
|---|---|---|
| Components | (wt %) | Supplier |
| Verinurad | 3.60 | AstraZeneca |
| HPMC 6 cps | 0.40 | Dow |
| Micro crystalline cellulose spheres 0.5-0.7 mm | 56.0 | JRS Pharma |
| EC 10 cps | 14.2 | Dow |
| PVP K30 | 5.80 | BASF |
| Sodium alginate Protanal LFR5/60 | 20.0 | Dupont |

### Example Formulation N

| | Quantity | |
|---|---|---|
| Components | (wt %) | Supplier |
| Verinurad | 3.38 | AstraZeneca |
| HPMC 6 cps | 0.38 | Dow |
| Micro crystalline cellulose spheres 0.5-0.7 mm | 52.5 | JRS Pharma |
| EC 10 cps | 13.3 | Dow |
| PVP K30 | 5.44 | BASF |
| Sodium alginate Protanal LFR5/60 | 25.0 | Dupont |

TABLE 1a
Process parameters used for coating of
pellets in Example Formulations A-H.

| | Alginate | EC:PVP | API |
|---|---|---|---|
| Process parameter | coat | coat | coat |
| Fluidization gas inlet temperature | 74-77 | 98-104 | 74-77 |
| (° C.) | | | |
| Fluidization gas flow (Nm³/h) | 9-12 | 9-11 | 10-11 |
| Spray rate (g/min) | 1.1-1.4 | 12-13 | 5 |
| Atomizer pressure (bar) | 1.4-1.5 | 2.2-2.4 | 1.2-1.3 |
| Atomizer gas flow (Nm³/h) | 1.6-1.8 | 2.3-2.5 | 1.4-1.5 |
| Batch size (g) | 10 | 20 | 100 |
| Solid content in coating solution | 3 | 6 | 10 |
| (wt %) | | | |
| Equipment | mini Glatt | mini Glatt | mini Glatt |

TABLE 1b
Process parameters used for coating of
pellets in Example Formulations I-N.

| | Alginate | EC:PVP | API |
|---|---|---|---|
| Process parameters | coat | coat | coat |
| Fluidization gas inlet temperature (° C.) | 71-77 | 100-102 | 70-84 |
| Fluidization gas flow (Nm³/h) | 9-15 | 40 | 40-42 |
| Spray rate (g/min) | 3.8-4.6 | 50 | 19-20 |
| Atomizer pressure (bar) | 2.1-2.3 | 3.7-4.8 | 2.3-2.6 |
| Atomizer gas flow (Nm³/h) | 2.2-2.3 | 3.7-4.7 | 2.5-3.0 |
| Batch size (g) | 20 | 200 | 450 |
| Solid content in coating solution (wt %) | 8 | 6 | 10 |
| Equipment | mini Glatt | LabCC | LabCC |

### Example 2

### Evaluation of Various Sodium Alginate Coatings on Inhibition of Alcohol-Induced Dose Dumping

The ability of various sodium alginate coatings to inhibit ADD of verinurad was evaluated according to the following procedure.

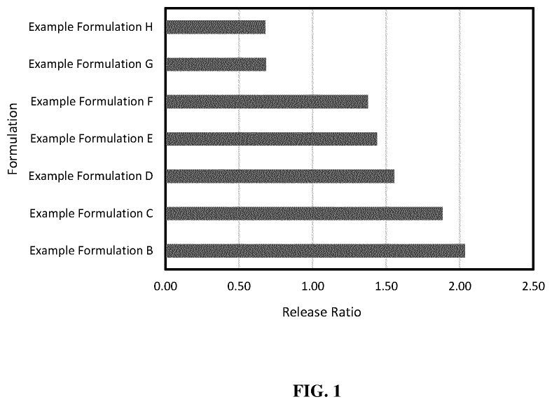

Verinurad (API) release from coated pellets was measured with a USP Apparatus 2 at 75 rpm. 500 mL of dissolution medium was used at 37° C. The control medium was 0.1M HCl (pH 1.1). Alcohol-containing medium was 0.1M HCl (pH 1.1) with 20 vol % ethanol. Approximately 100 mg of coated pellets was added to each vessel. The API released was quantified using an on-line UV-visible spectrophotometer system at wavelength 303 nm. Amount released, expressed as %, was calculated from normalized dissolution profiles assuming 100% released when profiles leveled out. The ratio of released amount of verinurad in 20% ethanol (EtOH) versus 0% EtOH at 2 hours was calculated for each of the formulations tested. The results are summarized in Table 2 and FIG. 1.

TABLE 2
Results from the evaluation of various alginate coatings
on inhibition of alcohol- induced dose dumping.

| | | | Release in | Release in | Release ratio |
|---|---|---|---|---|---|
| Example | Sodium | Amount | 0% EtOH at | 20% EtOH | 20% EtOH/0% |
| Formulation | Alginate | (wt %) | 2 h (%) | at 2 h (%) | EtOH at 2 h |
| A | None | N/A | 12.8 | 26.5 | 2.07 |
| B | None | N/A | 14.7 | 29.9 | 2.03 |
| C | Manucol LB | 25 | 11.1 | 20.8 | 1.88 |
| D | Protanal CR8133 | 25 | 9.3 | 14.4 | 1.56 |
| E | Manucol DH | 25 | 11.2 | 16.1 | 1.44 |
| F | Manucol LKX | 25 | 10.9 | 15.0 | 1.38 |
| G | Medium viscosity | 25 | 9.0 | 6.2 | 0.68 |
| H | Protanal LFR 5/60 | 25 | 11.5 | 7.8 | 0.68 |

The results in Table 2 and FIG. 1 demonstrate that Protanal LFR 5/60 and medium-viscosity sodium alginate were the most effective at inhibiting ADD of verinurad.

### Example 3

### Evaluation of the Amount of PROTANAL LFR 5/60 Sodium Alginate Layer on Inhibition of Alcohol-Induced Dose Dumping

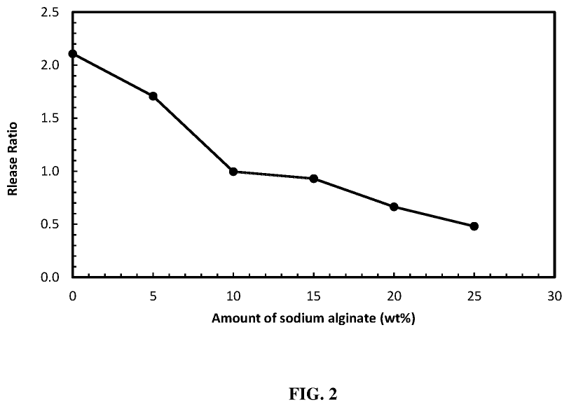

The impact of the amount of Protanal LFR 5/60 sodium alginate layer on inhibition of ADD of verinurad was evaluated according to the procedure described in Example 2. The results are summarized in Table 3 and FIG. 2.

TABLE 3
Results from the evaluation of different amounts of LFR 5/60
sodium alginate layer on alcohol-induced dose dumping.

| | | | Release in | Release in | Release ratio |
|---|---|---|---|---|---|
| Example | Sodium | Amount | 0% EtOH at | 20% EtOH | 20% EtOH/0% |
| Formulation | Alginate | (wt %) | 2 h (%) | at 2 h (%) | EtOH at 2 h |
| I | None | 0 | 12.1 | 25.5 | 2.11 |
| J | Protanal LFR 5/60 | 5 | 11.4 | 19.5 | 1.71 |
| K | Protanal LFR 5/60 | 10 | 10.9 | 10.9 | 1.00 |
| L | Protanal LFR 5/60 | 15 | 10.1 | 9.3 | 0.93 |
| M | Protanal LFR 5/60 | 20 | 12.4 | 8.2 | 0.66 |
| N | Protanal LFR 5/60 | 25 | 13.1 | 6.3 | 0.48 |

1. A pharmaceutical composition, wherein the pharmaceutical composition is a multiparticulate composition comprising a plurality of pellets, wherein each pellet comprises:
a core;
an API layer on the core, wherein the API layer comprises verinurad or a pharmaceutically acceptable salt thereof;
a rate-controlling polymer layer on the API layer; and
a sodium alginate layer on the release-rate controlling polymer layer.

2. The pharmaceutical composition of claim 1, wherein the rate-controlling polymer layer comprises ethyl cellulose.

3. The pharmaceutical composition of claim 1 or 2, wherein the rate-controlling polymer layer comprises polyvinylpyrrolidone.

4. The pharmaceutical composition of claim 1 or 2, wherein the rate-controlling polymer layer comprises hydroxypropyl cellulose.

5. The pharmaceutical composition of any one of the preceding claims, wherein the core comprises microcrystalline cellulose.

6. The pharmaceutical composition of any one of the preceding claims, wherein the API layer comprises verinurad.

7. The pharmaceutical composition of any one of the preceding claims, wherein the API layer further comprises hydroxypropyl methylcellulose.

8. The pharmaceutical composition of any one of the preceding claims, wherein the API layer further comprises a xanthine oxidase inhibitor.

9. The pharmaceutical composition of claim 8, wherein the xanthine oxidase inhibitor is allopurinol.

10. The pharmaceutical composition of any one claims 1 to 7, wherein the pharmaceutical composition further comprises a xanthine oxidase inhibitor.

11. The pharmaceutical composition of claim 10, wherein the xanthine oxidase inhibitor is allopurinol.

12. The pharmaceutical composition of any one of the preceding claims, wherein the sodium alginate has a glucuronic acid content between 65% and 75%, and a mannuronic acid content between 25% and 35%.

13. The pharmaceutical composition of any one of the preceding claims, wherein the amount of the sodium alginate layer is between 15 wt % and 25 wt %.

14. A pharmaceutical composition, wherein the pharmaceutical composition is a multiparticulate composition comprising a plurality of pellets, wherein each pellet comprises:
a core comprising microcrystalline cellulose;
an API layer on the core, wherein the API layer comprises verinurad or a pharmaceutically acceptable salt thereof;
a rate-controlling polymer layer on the API layer, wherein the rate-controlling polymer layer comprises ethyl cellulose and polyvinylpyrrolidone; and
a sodium alginate layer on the release-rate controlling polymer layer, wherein the sodium alginate has a glucuronic acid content between 65% and 75%, and a mannuronic acid content between 25% and 35%.

15. The pharmaceutical composition of claim 14, wherein the amount of the sodium alginate layer is between 15 wt % and 25 wt %.

16. The pharmaceutical composition of claim 14 or 15, wherein the API layer comprises verinurad.

17. The pharmaceutical composition of any one of claims 14 to 16, wherein the API layer further comprises a xanthine oxidase inhibitor.

18. The pharmaceutical composition of claim 17, wherein the xanthine oxidase inhibitor is allopurinol.

19. The pharmaceutical composition of any one of claims 14 to 16, wherein the pharmaceutical composition further comprises a xanthine oxidase inhibitor.

20. The pharmaceutical composition of claim 19, wherein the xanthine oxidase inhibitor is allopurinol.

21. A method of reducing serum uric acid levels in a human comprising administering a pharmaceutical composition of any one of the preceding claims to the human.

22. A method of treating or preventing hyperuricemia, gout, gouty arthritis, recurrent gout attacks, polycythemia, myeloid metaplasia, inflammatory arthritis, nephrolithiasis (kidney stones), joint inflammation, urolithiasis (formation of calculus in the urinary tract), deposition of urate crystals in joints, deposition of urate crystals in renal parenchyma, plumbism, hyperparathyroidism, psoriasis, sarcoidosis, Lesch-Nyhan syndrome, Kelley-Seegmiller syndrome, gout flare, tophaceous gout, chronic kidney disease, kidney failure, heart failure, hypertension, cardiovascular disease, coronary heart disease, or a combination thereof in a human comprising administering a pharmaceutical composition of any one of claims 1-20 to the human.

23. A method of treating or preventing chronic kidney disease in a human comprising administering a pharmaceutical composition of any one of claims 1-20 to the human.

24. A method of treating or preventing heart failure in a human comprising administering a pharmaceutical composition of any one of claims 1-20 to the human.
